(19) United States
(12) Patent Application Publication
(10) Pub. No.: US 2024/0424161 A1
(43) Pub. Date: Dec. 26, 2024

(54) MACHINE FOR TREATING A MEDICAL APPARATUS

(71) Applicant: Ecolab USA Inc., Saint Paul, MN (US)

(72) Inventors: Sébastien RUIZ, Saint-Victoret (FR); Vincent BRESSION, Gemenos (FR); Yolande MARTINEZ, La Ciotat (FR); Eric PENDARIES, La Ciotat (FR)

(21) Appl. No.: 18/647,405

(22) Filed: Apr. 26, 2024

Related U.S. Application Data
(60) Provisional application No. 63/499,090, filed on Apr. 28, 2023.

(51) Int. Cl. A61L 2/24

(52) U.S. Cl. CPC A61L 2/24; A61L 2202/122; A61L 2202/24

(57) ABSTRACT

A machine for treating a medical apparatus may include a treatment chamber having an enclosure and at least one opening configured to allow a medical apparatus to be loaded into or unloaded from the enclosure, a door configured to be moved according to a first direction between a closing position into which the opening is sealingly closed by the door and an opening position into which the door frees the opening, and an actuation system configured to be mechanically coupled to the door to move the door between the closing position and the opening position. In some examples, the actuation system is configured to disengage from the door when the door moves toward the closing position and a load applied to the door in opposition to the motion toward the closing position is equal or greater than a threshold value.

### RELATED APPLICATION

This application claims priority to U.S. Provisional Application No. 63/499,090, filed on Apr. 28, 2023, the entire contents of which are incorporated herein by reference.

### TECHNICAL FIELD

The disclosure relates to a machine for treating a medical apparatus embedded in a wall separating a first enclosure with a non-controlled atmosphere from a second enclosure with a controlled atmosphere. The disclosure falls under the domain of machines for treating medical apparatuses and more specifically endoscope washer-disinfector, also called automatic endoscope reprocessing (“AER”).

### BACKGROUND

An endoscope is a slender, tubular optical instrument used as a viewing system for examining an inner part of the body and, when used with an attached instrument, for biopsy or surgery. Decontamination systems can be used to reprocess previously-used medical devices, such as endoscopes such that the devices can be used again on a subsequent patient. During the decontamination process of an endoscope, the endoscope can be inserted into a chamber of a reprocessing machine and the internal channels of the endoscope connected to the machine to receive cleaning and/or disinfecting agents. For example, the reprocessing machine may provide a system of lines, pumps and valves for the purpose of feeding a cleaning and/or disinfecting agent to the endoscope placed in a chamber. The external surface of the endoscope may be sprayed with cleaning and/or disinfecting agent delivered through one or more spray assemblies inside of the chamber in which the endoscope is being reprocessed.

Some endoscope reprocessing machines include an unloading door and/or a loading door are manually operated to open and/or close the doors. In other configurations, the machine includes automated door(s) to minimize contacts with the machine, which could spread contaminants on the machine. Conventional systems of automatic doors are not, however, without drawbacks. For example, as the doors close, there may be an object or part of an operator obstructing the path of the door, which may cause damage or injury.

### SUMMARY

In some implementations, this disclosure describes a machine referred to as “pass-through” machine type, in that this machine is meant to be embedded in a wall separating a first enclosure with a non-controlled atmosphere from a second enclosure with a controlled atmosphere (for example the enclosure of an operating room or a clean room in which are stored medical apparatus, for which the controlled atmosphere is typically purified by filtration and/or extraction) from an enclosure with non-controlled atmosphere (for example a decontamination room in which a medical apparatus may be reprocessed) which may contain contaminants (for instance bacteria) in the air. This type of machine presents a loading door on the side of the first enclosure with the non-controlled atmosphere, allowing for example the loading of a contaminated medical apparatus from this side directly in an enclosure of the machine through a first opening of the machine, and an unloading door on the side of the second enclosure with controlled atmosphere, allowing for example to collect the disinfected medical apparatus from the side of the second enclosure in the enclosure of the machine through a second opening of the machine.

In different applications, a machine can have a single access door or multiple (e.g., two) access doors for loading and/or unloading a medical apparatus from the machine. In some examples, it is proposed that the machine for treating a medical apparatus includes:
at least one first treatment chamber having an enclosure and a least one first opening configured to allow a medical apparatus to be loaded into or unloaded from the enclosure,
at least one first door configured to be moved according to a first direction between a closing position into which the first opening is sealingly closed by the first door and an opening position into which the first door frees the first opening,
an actuation system configured to be mechanically coupled to the first door to move the first door between the closing position and the opening position,
   wherein the actuation system is configured to disengage from the first door when the first door moves toward the closing position and a load applied to the first door in opposition to the motion toward the closing position is equal or greater than a threshold value.

The actuation system may comprise:
a main actuator comprising a shaft adapted to move according to the first direction,
at least one first electromagnetic lock operatively connected to the shaft of the main actuator, said first electromagnetic lock being configured to be selectively locked with a counter element of the first door, and
   wherein the first electromagnetic lock is configured to be unlocked from the counter element of the first door during a motion of the first door toward the closing position when a load applied to the first door in opposition to the motion toward the closing position is equal or greater than the threshold value.

The threshold value may correspond to a magnetic force locking the first electromagnetic lock to the counter element.

The actuation system may be configured such that, when the first door moves toward the closing position and the load applied to the first door in opposition to the motion toward the closing position is equal or greater than the threshold value, the shaft is configured to pull the first electromagnetic lock away from the counter element thereby unlocking the first electromagnetic lock from the counter element.

The first electromagnetic lock may be configured to bear against the counter element of the first door according to the first direction such that the first door is pushed by the first electromagnetic lock for being moved toward the opening position and is pulled by the first electromagnetic lock for being moved toward the closing position.

The actuation system may be automatic.

The first direction may extend horizontally.

The first treatment chamber may comprise a second opening configured to allow a medical apparatus to be loaded into or unloaded from the enclosure, the first opening and the second opening may be opposite from each other according to a second direction, the machine for treating a medical apparatus further comprising a second door configured to be moved horizontally between a closing position into which the second opening is sealingly closed by the second door and an opening position into which the second door frees the second opening, the actuation system being configured be mechanically coupled to the second door to move the second door independently between the closing position and the opening position, wherein the actuation system is configured to disengage from the second door when the second door moves toward the closing position and a load applied to the second door in opposition to the motion toward the closing position is equal or greater than the threshold value.

The actuation system may comprise a second electromagnetic lock operatively connected to the shaft of the main actuator, said second electromagnetic lock being configured to be selectively locked with a counter element of the second door, wherein the second electromagnetic lock is configured to be unlocked from the counter element of the second door during a motion of the second door toward the closing position when a load applied to the second door in opposition to the motion toward the closing position is equal or greater than the threshold value.

The actuation system may comprise a cross bar extending along the second direction between a first end and a second end, the cross bar being operatively connected at a middle portion to the shaft of the main actuator, the first electromagnetic lock and the second electromagnetic lock being slidably connected along the second direction respectively at the first end and the second end of the cross bar between an extended position in which the first electromagnetic lock and the second electromagnetic lock are configured to engage with the corresponding counter element of the first door and the second door, and a retracted position in which the first electromagnetic lock and the second electromagnetic lock are located remotely from the corresponding counter element of the first door and the second door.

The second electromagnetic lock may be configured to bear against the counter element of the second door according to the first direction such that the second door is pushed by the second electromagnetic lock for being moved toward the opening position and is pulled by the second electromagnetic lock for being moved toward the closing position.

In the extended position, the first electromagnetic lock and the second electromagnetic lock may be facing along the first direction the corresponding counter element of the first door and the second door, and in the retracted position, the first electromagnetic lock and the second electromagnetic lock may be located offset with the corresponding counter element of the first door and the second door along the second direction.

The actuation system may comprise:
a first bracket and a second bracket on which are respectively operatively connected the first electromagnetic lock and the second electromagnetic lock, the first bracket and the second bracket being slidably connected along the second direction respectively at the first end and the second end of the cross bar,
a first auxiliary actuator having a body operatively connected to the first end of the cross bar and a rod adapted to be moved according to the second direction, a distal end of the rod being operatively connected to the first bracket in order to move the first electromagnetic between the extended position and the retracted position,
a second auxiliary actuator having a body operatively connected to the second end of the cross bar and a rod adapted to be moved move according to the second direction, a distal end of the rod being operatively connected to the second bracket in order to move the second electromagnetic lock between the extended position and the retracted position.

The machine for treating a medical apparatus may further comprise at least one first treatment circuit 25 adjacent to the first treatment chamber along the first direction and which is adapted to treat a medical apparatus disposed in the first treatment chamber by immersion or by spraying a disinfectant solution taken from a reservoir.

The machine for treating a medical apparatus may further comprise a frame, said frame comprising at least one first rail extending according to the first direction into which the first door slides between the closing position and the opening position.

The frame may be adapted to be embedded in a wall separating a controlled atmosphere enclosure from an enclosure with a non-controlled atmosphere.

One or more of the doors may be configured to be locked with the frame when in the closing position.

The machine for treating a medical apparatus may further comprise a programmable controller for controlling the actuation system to open and/or close one or more of the doors of the machine.

The machine for treating a medical apparatus may further comprise a user interface to allow an operator to actuate one or more of the doors of the machine.

### BRIEF DESCRIPTION OF DRAWINGS

Other features, details and advantages will be shown in the following detailed description and on the figures, on which:

FIG. 1 is schematic overview of the machine for treating a medical apparatus according to the present disclosure.

FIG. 2 is a schematic perspective view from one side of the machine for treating a medical apparatus according to the present disclosure.

FIG. 3 is a schematic perspective view from another side of the machine for treating a medical apparatus according to the present disclosure.

FIG. 4 is a schematic perspective view of a set of four doors and an actuation system of the machine for treating a medical apparatus according to the present disclosure.

FIG. 5 shows a side view of FIG. 4.

FIG. 6 is a larger scale schematic view of the zone I outlined in dotted lined in FIG. 5.

FIG. 7 is another larger scale schematic view of the zone I outlined in dotted lined in FIG. 5 according to another perspective.

FIG. 8 is a larger scale schematic view of the zone II outlined in dotted lined in FIG. 5.

### DETAILED DESCRIPTION

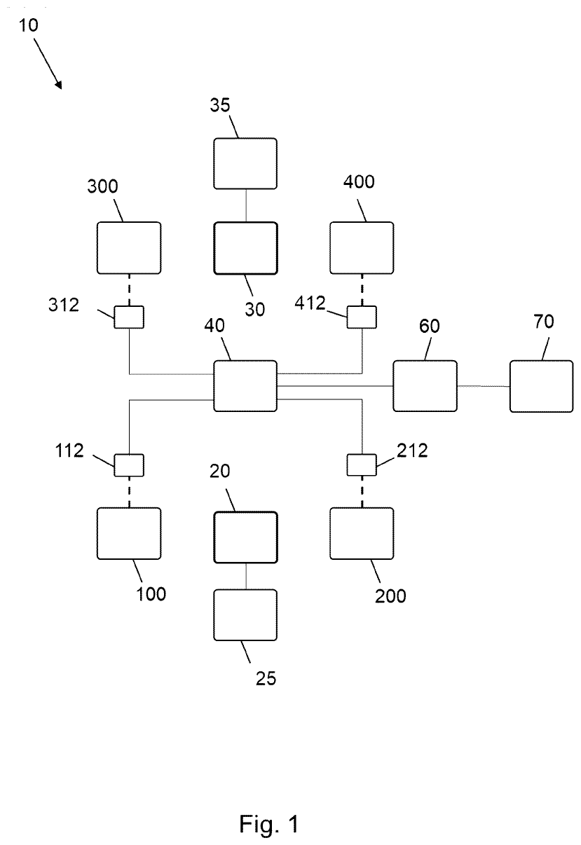

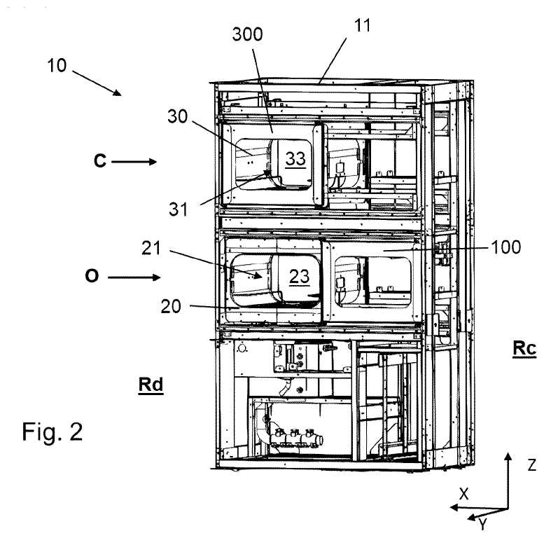

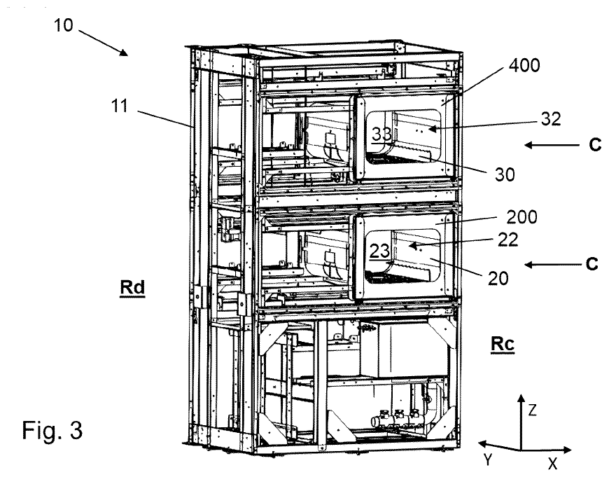

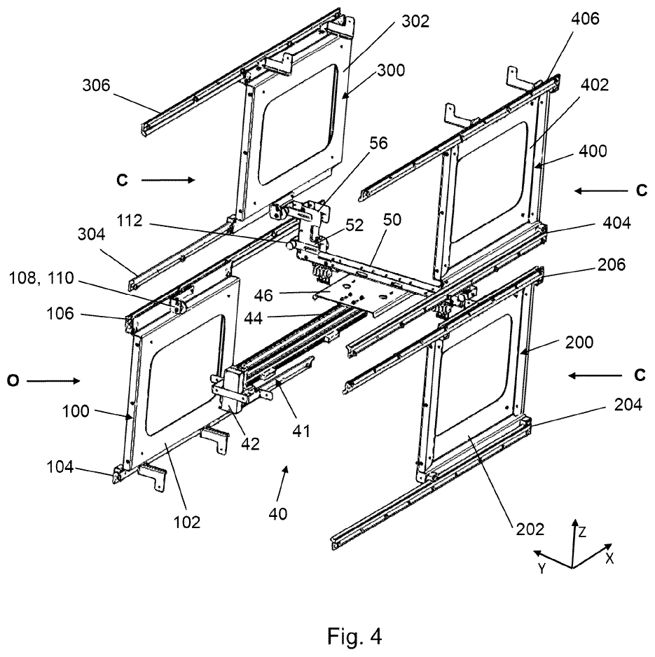

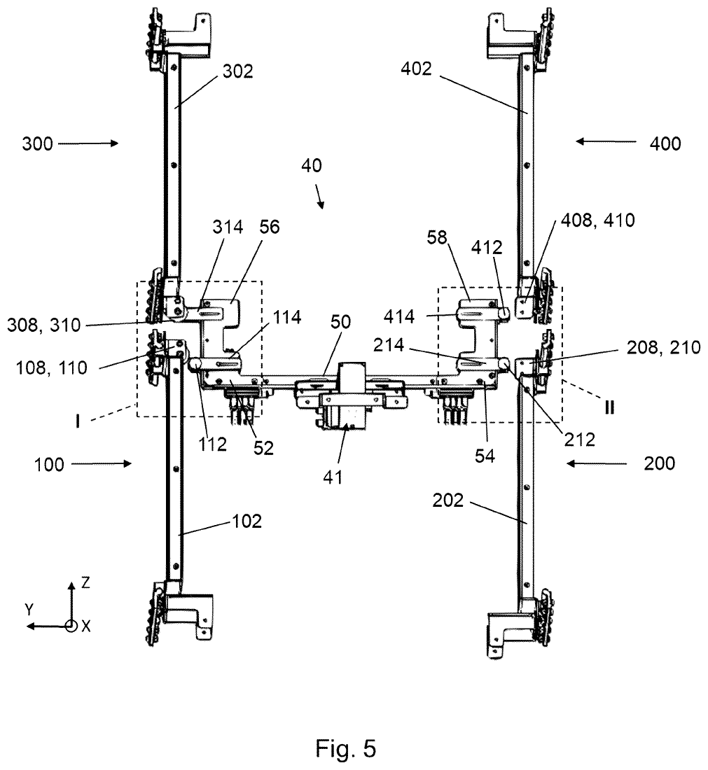

FIGS. 1 to 5 show an embodiment of a machine 10 for treating a medical apparatus according to the present disclosure.

The machine 10 for treating a medical apparatus comprises at least one first treatment chamber 20 having an enclosure 23 and a least one first opening 21 configured to allow a medical apparatus to be loaded into or unloaded from the enclosure 23. The machine 10 for treating a medical apparatus comprises also comprises at least one first door 100 configured to be moved according to a first direction X between a closing position C into which the first opening 21 is sealingly closed by the first door 100 and an opening position O into which the first door 100 frees the first opening. The first direction X may extend horizontally.

The machine 10 for treating a medical apparatus comprises an actuation system 40 configured to be mechanically coupled to the first door 100 to move the first door 100 between the closing position C and the opening position O. The actuation system 40 is configured to disengage from the first door 100 when the first door 100 moves toward the closing position C and a load applied to the first door 100 in opposition to the motion toward the closing position C is equal or greater than a threshold value. The first door 100 may be automatically opened and closed by the actuation system 40 which minimizes physical contact with the machine 10 and helps avoid the transmission of contaminants. The actuation system 40 may be automatic.

The disengagement (or uncoupling) of the actuation system 40 from the first door 100 causes the door to stop, thus preventing injury or damage. The machine 10 for treating a medical apparatus has an improved safety for an operator manipulating a medical apparatus such as an endoscope through the first opening 21, especially if it occurs that the first door 100 moves toward its closing position C.

The threshold value may be comprised between 50 N and 150 N, in particular for a steady load. Alternatively, the threshold value may be comprised between 150 N and 250 N for a load applied during less than 0.75 s. A steady load may be applied for a time duration greater than 0.75 S.

The actuation system 40 may comprise a main actuator 41. The main actuator 41 comprises a shaft 44 adapted to move according to the first direction X. The main actuator 41 may comprise an electric motor. The main actuator may comprise a worm screw integral with or operatively connected to the shaft 44. The electric motor may be adapted to move the worm screw according to the first direction X, for example by means of at least one gear.

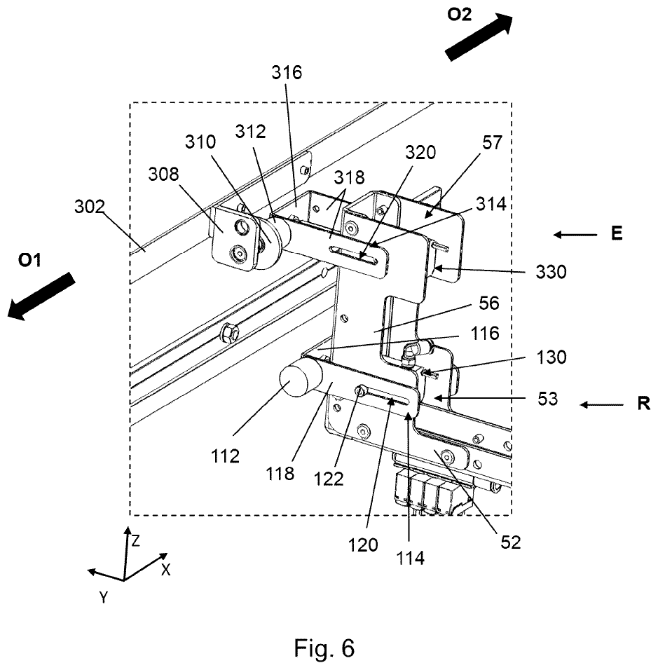

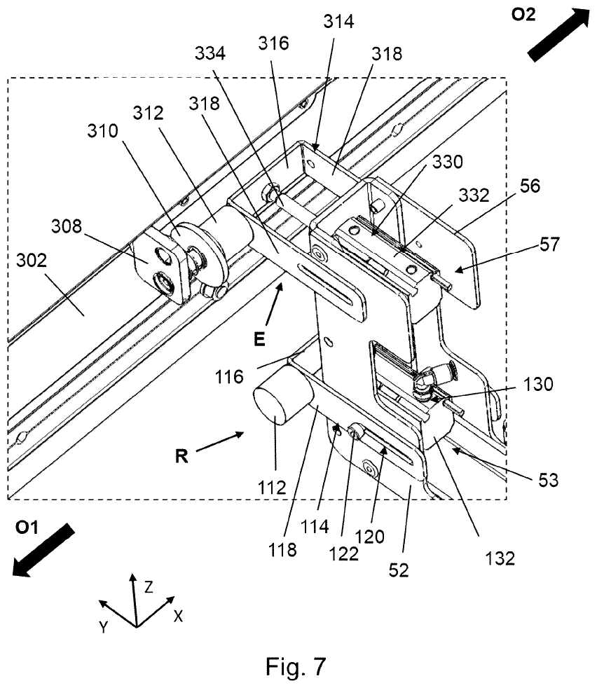

As illustrated in FIGS. 6 and 7, the actuation system 40 may comprise at least one first electromagnetic lock 112 operatively connected to the shaft 44 of the main actuator 41, said first electromagnetic lock 112 being configured to be selectively locked with a counter element 110 of the first door 100. The first electromagnetic lock 112 may be configured to be unlocked from the counter element 110 of the first door 100 during a motion of the first door 100 toward the closing position C when a load applied to the first door 100 in opposition to the motion toward the closing position C is equal or greater than the threshold value.

Locking the first electromagnetic lock 112 with the corresponding counter element 110 of the first door 100 allows operating the first door 100 between the closing position C and the opening position O by the actuation system 40. On the other hand, the shaft 44 of the main actuator 41 may be operated independently from the first door 100 when the first electromagnetic lock 112 is unlocked with the counter element 110 of the first door 100.

The stroke of the shaft 44 along the first direction X may correspond to the distance covered by the first door 100 along the first direction X between the closing position C and the opening position O.

By being “operatively connected”, it is meant that two parts are fixed to each other, either directly or by the means of intermediate parts and in some instance could also be said “rigidly connected”.

The threshold value may correspond to a magnetic force locking the first electromagnetic lock 112 to the counter element 110.

In other words, the electromagnetic lock may hold a force a load up to the threshold value, thus causing the disengagement between the actuation system 40 and the first door 100 if a load applied to the first door 100 in opposition to the motion toward the closing position C is equal or greater than the threshold value and also causing the door to stop. The actuation system 40 provides a safety system at a limited cost through the strength of the electromagnetic lock.

The actuation system 40 may be configured such that, when the first door 100 moves toward the closing position C and the load applied to the first door 100 in opposition to the motion toward the closing position C is equal or greater than the threshold value, the shaft 44 is configured to pull the first electromagnetic lock 112 away from the counter element 110 thereby unlocking the first electromagnetic lock 112 from the counter element 110.

The first electromagnetic lock 112 may be configured to bear against the counter element 110 of the first door 100 according to the first direction X such that the first door 100 is pushed by the first electromagnetic lock 112 for being moved toward the opening position O and is pulled by the first electromagnetic lock 112 for being moved toward the closing position C.

Therefore, for each door moving toward its closing position C, if it occurs that a load applied on the door in opposition to the motion toward the closing position C is equal or greater than the threshold value, the corresponding electromagnetic lock of the actuation system 40 is unlocked (in other words uncoupled or disengaged) from the respective counter element of the door which stops the motion of the door toward its closing position C.

The first treatment chamber 20 may comprise a second opening 22 configured to allow a medical apparatus to be loaded into or unloaded from the enclosure 23, the first opening 21 and the second opening 22 may be opposite from each other according to a second direction Y, the machine 10 for treating a medical apparatus further comprising a second door 200 configured to be moved horizontally between a closing position C into which the second opening 22 is sealingly closed by the second door 200 and an opening position O into which the second door 200 frees the second opening 22, the actuation system 40 being configured be mechanically coupled to the second door 200 to move the second door 200 independently between the closing position C and the opening position O. The actuation system 40 may be configured to disengage from the second door 200 when the second door 200 moves toward the closing position C and a load applied to the second door 200 in opposition to the motion toward the closing position C is equal or greater than the threshold value.

The machine 10 for treating a medical apparatus may be referred as a “pass-through” machine type, for instance enabling passing a medical apparatus form an uncontrolled atmosphere zone to a controlled atmosphere zone.

The second door 200 is also automatically opened and closed by the actuation system 40 which minimizes physical contact with the machine 10 and helps avoid the transmission of contaminants. The disengagement of the actuation system 40 from the second door 200 causes the second door 200 to stop, thus preventing injury or damage. The machine 10 for treating a medical apparatus has an improved safety for an operator manipulating medical apparatus through the second opening 22, especially if it occurs that the second door 200 moves toward its closing position C.

The second direction Y may extend horizontally. The first direction X and the second direction Y may be perpendicular to each other. In other words, the first opening 21 and the second opening 22 may be spaced apart from each other according to the second direction Y.

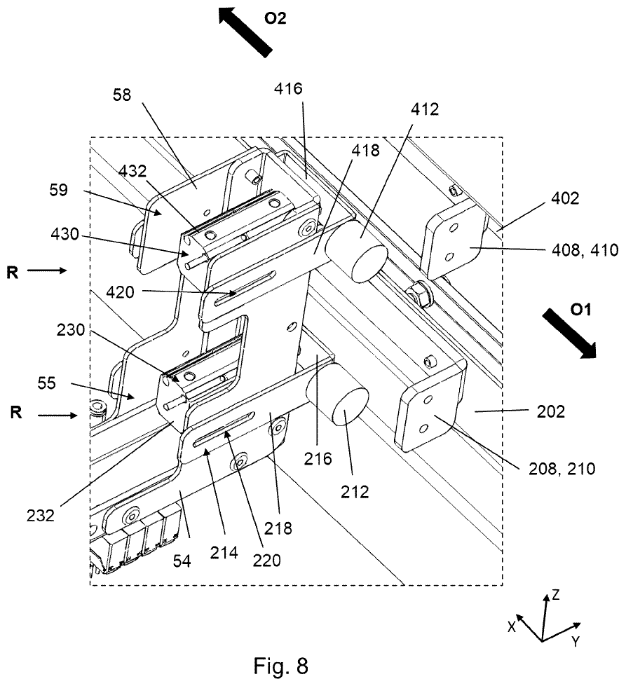

As better illustrated in FIGS. 8, he actuation system 40 may comprise a second electromagnetic lock 212 operatively connected to the shaft 44 of the main actuator, said second electromagnetic lock 212 being configured to be selectively locked with a counter element 210 of the second door 200. The second electromagnetic lock 212 is configured to be unlocked from the counter element 210 of the second door 200 during a motion of the second door 200 toward the closing position C when a load applied to the second door 200 in opposition to the motion toward the closing position C is equal or greater than the threshold value.

The actuation system 40 advantageously comprises a single main actuator 41, which may limit the cost of the machine 10.

Locking the second electromagnetic lock 212 with the corresponding counter element 210 of the second door 200 allows operating the second door 200 between the closing position C and the opening position O by the actuation system 40. On the other hand, the shaft 44 of the main actuator 41 may be operated independently from the second door 200 when the second electromagnetic lock 212 is unlocked with the counter element 210 of the second door 200. Therefore, the first door 100 and the second door 200 may be operated by the single main actuator 41 between their respective closing position C and opening position O, either together or independently from each other.

The main actuator 41 may be at least in part disposed between the first door 100 and the second door 200 according to the second direction Y.

The threshold value may correspond to a magnetic force locking the second electromagnetic lock 212 to the corresponding counter element 210.

The actuation system 40 may be configured such that, when the second door 200 moves toward the closing position C and the load applied to the second door 200 in opposition to the motion toward the closing position C is equal or greater than the threshold value, the shaft 44 is configured to pull the second electromagnetic lock 212 away from the counter element 210 thereby unlocking the second electromagnetic lock 212 from the counter element 210 of the second door 200.

The actuation system 40 may comprise a cross bar 50 extending along the second direction Y between a first end 52 and a second end 54, the cross bar 50 being operatively connected at a middle portion to the shaft 44 of the main actuator, the first electromagnetic lock 112 and the second electromagnetic lock 212 being slidably connected along the second direction Y respectively at the first end 52 and the second end 54 of the cross bar 50 between an extended position E in which the first electromagnetic lock 112 and the second electromagnetic lock 212 are configured to engage with the corresponding counter element 110, 210 of the first door 100 and the second door 200, and a retracted position R in which the first electromagnetic lock 112 and the second electromagnetic lock 212 are located remotely from the corresponding counter element 110, 210 of the first door 100 and the second door 200.

The actuation system 40 may comprise a plate 46 extending horizontally. The plate 46 may be fixed at a distal end of the shaft 44 of the main actuator 41 and the middle portion of the cross bar 50 may be fixed on the plate 46.

The second electromagnetic lock 212 may be configured to bear against the counter element 210 of the second door 200 according to the first direction X such that the second door 200 is pushed by the second electromagnetic lock 212 for being moved toward the opening position O and is pulled by the second electromagnetic lock 212 for being moved toward the closing position C.

In the extended position E, the first electromagnetic lock 112 and the second electromagnetic lock 212 may be facing along the first direction X the corresponding counter element 110, 210 of the first door 100 and the second door 200. In the retracted position R, the first electromagnetic lock 112 and the second electromagnetic lock 212 may be located offset with the corresponding counter element 110, 210 of the first door 100 and the second door 200 along the second direction Y. In other words, in the retracted position R, the first electromagnetic lock 112 and the second electromagnetic lock 212 may not face the corresponding counter element 110, 210 of the first door 100 and the second door 200 along the first direction X.

The actuation system 40 may comprise:
a first bracket 114 and a second bracket 214 on which are respectively operatively connected the first electromagnetic lock 112 and the second electromagnetic lock 212, the first bracket 114 and the second bracket 214 being slidably connected along the second direction Y respectively at the first end 52 and the second end 54 of the cross bar 50,
a first auxiliary actuator 130 having a body 132 operatively connected to the first end 52 of the cross bar 50 and a rod 134 adapted to be moved according to the second direction Y, a distal end of the rod 134 being operatively connected to the first bracket 114 in order to move the first electromagnetic between the extended position E and the retracted position R,
a second auxiliary actuator 230 having a body 232 operatively connected to the second end 54 of the cross bar 50 and a rod 234 adapted to be moved move according to the second direction Y, a distal end of the rod 234 being operatively connected to the second bracket 214 in order to move the second electromagnetic lock 212 between the extended position E and the retracted position R.

The first bracket 114 and the second bracket 214 may be U-shaped. Each of the U-shaped first bracket 114 and second bracket 214 may comprise a central portion 116, 216 extending along the first direction X. Each of the U-shaped first bracket 114 and second bracket 214 may comprise a pair of branches 118, 218 each connected at one end of the central portion 116, 216 and extending along the second direction Y. Branches 118 of the U-shaped first bracket 114 may be on both sides of the first end 52 of the cross bar 50 and branches 218 of the U-shaped second bracket 214 may be on both sides of the second end 54 of the cross bar 50. Each branch 118 of the U-shaped first bracket 114 may comprise an elongated hole 120 along the second direction Y in which is received a corresponding protrusion 122 extending from the first end 52 of the cross bar 50. Each branch 218 of the U-shaped second bracket 214 may comprise an elongated hole 220 along the second direction Y in which is received a corresponding protrusion extending from the second end 54 of the cross bar 50.

The distal end of the rod 134 of the first auxiliary actuator 130 may be operatively connected to the central portion 116 of the U-shaped first bracket 114. The distal end of the rod 234 of the second auxiliary actuator 230 may be operatively connected to the central portion 216 of the U-shaped second bracket 214.

The first end 52 of the cross bar 50 may define a recess 53 or a housing in which is at least in part received the body 132 of the first auxiliary actuator 130. The second end 54 of the cross bar 50 may define a recess 55 or a housing in which is at least in part received the body 232 of the second auxiliary actuator 230.

The machine 10 for treating a medical apparatus may further comprise at least one first treatment circuit adjacent to the first treatment chamber 20 along the first direction X and which is adapted to treat a medical apparatus disposed in the first treatment chamber 20 by immersion or by spraying a disinfectant solution taken from a reservoir. The reservoir may be inside or outside of the machine 10. One or more of the treatment circuits may comprise circuitry, one or more pumps and/or one or more solenoid valve.

The machine 10 for treating a medical apparatus according may further comprise:
a second treatment chamber 30 disposed along a third direction Z extending vertically above the first treatment chamber 20, the second treatment chamber 30 having an enclosure 33 and a least a first opening 31 and second opening 32 each configured to allow a medical apparatus to be loaded into or unloaded from the enclosure 33,
a third door 300 configured to be moved horizontally according to the first direction X between a closing position C into which the first opening 31 of the second chamber is sealingly closed by the third door 300 and an opening position O into which the third door 300 frees the first opening 31 of the second treatment chamber 30,
a fourth door 400 configured to be moved horizontally according to the first direction X between a closing position C into which the second opening 32 of the second chamber is sealingly closed by the fourth door 400 and an opening position O into which the fourth door 400 frees the second opening 32 of the second treatment chamber 30.

The actuation system 40 may be configured be mechanically coupled to the third door 300 and/or to the fourth door 400 to move the third door 300 and/or the fourth door 400 independently between the closing position C and the opening position O. The actuation system 40 may be configured to disengage from the third door 300 when the third door 300 moves toward the closing position C and a load applied to the third door 300 in opposition to the motion toward the closing position C is equal or greater than a threshold value. The actuation system 40 may be configured to disengage from the fourth door 400 when the fourth door 400 moves toward the closing position C and a load applied to the fourth door 400 in opposition to the motion toward the closing position C is equal or greater than a threshold value.

The machine 10 for treating a medical apparatus has an improved safety for an operator manipulating medical apparatus through the first opening 31 or the second opening 32 of the second treatment chamber 30, especially if it occurs that the third door 300 and/or the fourth door 400 moves toward its closing position C.

The actuation system 40 may comprise a third electromagnetic lock 312 operatively and a fourth electromagnetic lock 412 connected to the shaft 44 of the main actuator.

Said third electromagnetic lock 312 may be configured to be selectively locked with a counter element 310 of the third door 300. The third electromagnetic lock 312 may be configured to be unlocked from the counter element 310 of the third door 300 during the motion of the third door 300 toward the closing position C when a load applied to the third door 300 in opposition to the motion toward the closing position C is equal or greater than the threshold value.

Locking the third electromagnetic lock 312 with the corresponding counter element 310 of the third door 300 allows operating the third door 300 between the closing position C and the opening position O by the actuation system 40. On the other hand, the shaft 44 of the main actuator 41 may be operated independently from the third door 300 when the third electromagnetic lock 312 is unlocked with the counter element 310 of the third door 300. Therefore, the first door 100, the second door 200 and the third door 300 may be operated by the single main actuator 41 between their respective closing position C and opening position O, either together or independently from each other.

The fourth electromagnetic lock 412 may be configured to be selectively locked with a counter element 410 of the fourth door 400. The fourth electromagnetic lock 412 may be configured to be unlocked from the counter element 410 of the fourth door 400 during the motion of the fourth door 400 toward the closing position C when a load applied to the fourth door 400 in opposition to the motion toward the closing position C is equal or greater than the threshold value.

Locking the fourth electromagnetic lock 412 with the corresponding counter element 410 of the fourth door 400 allows operating the fourth door 400 between the closing position C and the opening position O by the actuation system 40. On the other hand, the shaft 44 of the main actuator 41 may be operated independently from the fourth door 400 when the fourth electromagnetic lock 412 is unlocked with the counter element 410 of the fourth door 400. Therefore, the first door 100, the second door 200, the third door 300 and the fourth door 400 may be operated by the single main actuator 41 between their respective closing position C and opening position O, either together or independently from each other.

The threshold value may correspond to a magnetic force locking the third electromagnetic lock 312 or the fourth electromagnetic lock 412 to the corresponding counter element 310, 410.

The actuation system 40 may be configured such that, when the third door 300 moves toward the closing position C and the load applied to the third door 300 in opposition to the motion toward the closing position C is equal or greater than the threshold value, the shaft 44 is configured to pull the third electromagnetic lock 312 away from the counter element 310 thereby unlocking the third electromagnetic lock 312 from the counter element 310 of the third door 300.

The actuation system 40 may be configured such that, when the fourth door 400 moves toward the closing position C and the load applied to the fourth door 400 in opposition to the motion toward the closing position C is equal or greater than the threshold value, the shaft 44 is configured to pull the fourth electromagnetic lock 412 away from the counter element 410 thereby unlocking the fourth electromagnetic lock 412 from the counter element 410 of the fourth door 400.

The actuation system 40 may comprise a first branch 56 and a second branch 58 extending along the third direction Z respectively from the first end 52 and the second end 54 of the cross bar 50. The first branch 56 and the second branch 58 may be integral with or operatively connected to the cross bar 50.

The third electromagnetic lock 312 and the fourth electromagnetic lock 412 may be slidably connected along the second direction Y respectively to the first branch 56 and the second brand, between an extended position E in which the third electromagnetic lock 312 and the fourth electromagnetic lock 412 are configured to engage with the corresponding counter element 310, 410 of the third door 300 and the fourth door 400, and a retracted position R in which the third electromagnetic lock 312 and the fourth electromagnetic lock 412 are located remotely from the corresponding counter element 310, 410 of the third door 300 and the fourth door 400.

The third electromagnetic lock 312 is configured to bear against the counter element 310 of the third door 300 according to the first direction X such that the third door 300 is pushed by the third electromagnetic lock 312 for being moved toward the opening position O and is pulled by the third electromagnetic lock 312 for being moved toward the closing position C.

The fourth electromagnetic lock 412 is configured to bear against the counter element 410 of the fourth door 400 according to the first direction X such that the fourth door 400 is pushed by the third electromagnetic lock 312 for being moved toward the opening position O and is pulled by the fourth electromagnetic lock 412 for being moved toward the closing position C.

In the extended position E, the third electromagnetic lock 312 and the fourth electromagnetic lock 412 are facing along the first direction X the corresponding counter element 310, 410 of the third door 300 and the fourth door 400, and where in the retracted position R, the third electromagnetic lock 312 and the fourth electromagnetic lock 412 are located offset with the corresponding counter element 310, 410 of the third door 300 and the fourth door 400 along the second direction Y.

The actuation system 40 may comprise:
a third bracket 314 and a fourth bracket 414 on which are respectively operatively connected the third electromagnetic lock 312 and the fourth electromagnetic lock 412, the third bracket 314 and the fourth bracket 414 being slidably connected along the second direction Y respectively to the first branch 56 and the second branch 58,
a third auxiliary actuator 330 having a body 332 operatively connected to the first branch 56 and a rod 334 adapted to be moved according to the second direction Y, a distal end of the rod 334 being operatively connected to the third bracket 314 in order to move the third electromagnetic between the extended position E and the retracted position R,
a fourth auxiliary actuator 430 having a body 432 operatively connected to the second branch 58 and a rod 434 adapted to be moved according to the second direction Y, a distal end of the rod 434 being operatively connected to the fourth bracket 414 in order to move the fourth electromagnetic lock 412 between the extended position E and the retracted position R.

One or more of the auxiliary actuators 130, 230, 330, 430 may be a pneumatic cylinder.

The third bracket 314 and the fourth bracket 414 may be U-shaped. Each of the U-shaped third bracket 314 and fourth bracket 414 may comprise a central portion 316, 416 extending along the first direction X. Each of the U-shaped third bracket 314 and fourth bracket 414 may comprise a pair of branches 318, 418 each connected at one end of the central portion 316, 416 and extending along the second direction Y. Branches 318, 418 of the U-shaped third bracket 314 may be on both sides of the first branch 56 and branches 318, 418 of the U-shaped second bracket 214 may be on both sides of the second branch 58. Each branch 318 of the U-shaped third bracket 314 may comprise an elongated hole 320 along the second direction Y in which is received a corresponding protrusion extending from the first branch 56. Each branch 418 of the U-shaped fourth bracket 414 may comprise an elongated hole 420 along the second direction Y in which is received a corresponding protrusion extending from the second branch 58.

The distal end of the rod 334 of the third auxiliary actuator 330 may be operatively connected to the central portion 316 of the U-shaped third bracket 314. The distal end of the rod 434 of the fourth auxiliary actuator 430 may be operatively connected to the central portion 416 of the U-shaped fourth bracket 414.

The first branch 56 may define a recess 57 or a housing in which is at least in part received the body 332 of the third auxiliary actuator 330. The second branch 58 may define a recess 59 or a housing in which is at least in part received the body 432 of the fourth auxiliary actuator 430.

The machine 10 for treating a medical apparatus may comprise a second treatment circuit 35 adjacent to the second treatment chamber 30 along the first direction X and which is adapted to treat a medical apparatus in the second treatment chamber 30 by immersion or by spraying a disinfectant solution taken from a reservoir.

The main actuator 41, the plate, the cross bar 50 may at least in part disposed between the first treatment chamber 20 and the second treatment chamber 30 according to the third direction Z. The machine 10 is advantageously compact.

The machine 10 for treating a medical apparatus may comprise one or more additional treatment chambers and/or treatment circuit disposed along a third direction Z extending vertically above the second treatment chamber 30 and which may be identical to the second treatment chamber 30.

The machine 10 for treating a medical apparatus may further comprise a frame 11, said frame 11 comprising at least one first rail 104, 106 extending according to the first direction X into which the first door 100 slides between the closing position C and the opening position O. Accordingly, one or more of the doors 100, 200, 300, 400 may be slidably engaged in a corresponding rail 104, 106, 204, 206, 304, 306, 404, 406 of the frame 11 in order to be guided along the first direction X between the closing position C and the opening position O. One or more of the doors 100, 200, 300, 400 may be slidably engaged in a corresponding lower rail 104, 204, 304, 404 and a corresponding upper rail 106, 206, 306, 406 of the frame 11 in order to be guided along the first direction X between the closing position C and the opening position O.

The main actuator 41 may comprise a body 42 operatively fixed to the frame 11.

The frame 11 may be adapted to be embedded in a wall separating a first enclosure Rc with controlled atmosphere from a second enclosure Rd with a non-controlled atmosphere.

One or more of the doors 100, 200, 300, 400 may be locked in the closing position C relatively to the frame 11. The frame 11 may comprise one or more auxiliary electromagnetic lock. One or more of the auxiliary electromagnetic locks 112, 212, 312, 412 may be adapted to cooperate with an auxiliary counter element of a respective one among the one or more doors being in the closing position C.

The machine 10 for treating a medical apparatus may further comprise a programmable controller 60 for controlling the actuation system 40 in order to open and/or close one or more of the doors 100, 200, 300, 400 of the machine 10. The programmable controller may control the electric motor of the main actuator 41. The programmable controller may also control the one or more of the treatment circuits to implement the disinfection of medical apparatus in the corresponding treatment chambers.

The machine 10 for treating a medical apparatus may further comprise a user interface 70 to allow an operator to actuate one or more of the doors 100, 200, 300, 400 of the machine 10. The opening/closing controls made at the user interface may be received by the programmable controller to activate the actuation system 40. The user interface may be touchless to ensure completely non-contact process.

It is understood that one or more of the doors may move toward the opening position O according to a first orientation O1 along the first direction X and toward the closing position C according to second orientation O2 along the first direction X. The first direction X may be referred as the first movement direction.

One or more of the doors 100, 200, 300, 400 may comprise a panel 102, 202, 302, 402 having a form adapted to sealingly close the corresponding opening 21, 22, 31, 32. One or more of the doors 100, 200, 300, 400 may comprise a bracket 108, 208, 308, 408 operatively connected to the panel 102, 202, 302, 402 on which is mounted the corresponding one or more counter element (i.e. counter elements 110, 210, 310, 410 configured for driving the corresponding door by means of the main actuator 41 and/or auxiliary counter element). One or more of the counter elements may be a plate. One or more of the counter elements may be made of a magnetizable material which does not corrode, for instance stainless steel, mild steel with surface treatment (electro-zincing).

The electromagnetic locks (i.e. electromagnetic locks configured for driving the corresponding door by means of the main actuator 41 and/or auxiliary electromagnetic locks) may also be called electromagnetic lock suction cups.

One or more of the electromagnetic locks may hold a force a load up to the threshold value, thus causing the disengagement between the actuation system 40 and the corresponding door if a load applied to the corresponding door in opposition to a motion toward the closing position C is equal or greater than the threshold value and also causing the door to stop. The actuation system 40 provides a safety system at a limited cost through the strength of the electromagnetic lock.

One or more of the electromagnetic locks (i.e. electromagnetic locks 112, 212, 312, 412 configured for driving the corresponding door by means of the main actuator 41 and/or auxiliary electromagnetic locks) may ensure locking with the corresponding counter element without being supplied with electricity from an external power source. In that case, the electromagnetic lock may be unlocked with the corresponding counter element by being supplied with electricity from an external power source. Such an electromagnetic lock does not heat up while operating. Alternatively, one or more of the electromagnetic locks may be supplied with electricity from an external power supply to ensure locking with the counter element. Such an electromagnetic lock provides a stronger locking.

One or more of the doors 100, 200, 300, 400 may be equipped with one or more limit sensor switch to collect positions of the corresponding door along the first direction X, at least to collect data when the door is in the closing position C or the opening position O. The shaft 44 of the actuation system 40 may be equipped with one or more limit sensor switch to collect positions of the shaft 44 along the first direction X, at least to collect data when the shaft 44 is in an end position. The interrelationship between data retrieved from limit sensor switch(es) of one of the doors 100, 200, 300, 400 and limit sensor switches of the actuation system 40 may indicate the unlocking of the corresponding electromagnetic lock during the motion of the door toward the closing position C and therefore indicate that the door is incompletely closed. One or more of the auxiliary actuators 130, 230, 330, 430 may be equipped with one or more limit sensor switch to collect data indicating which one(s) of the one or more doors 100, 200, 300, 400 is (are) connected to the actuation system 40.

1. A machine for treating a medical apparatus comprising:
at least one first treatment chamber having an enclosure and a least one first opening configured to allow a medical apparatus to be loaded into or unloaded from the enclosure;
at least one first door configured to be moved according to a first direction between a closing position into which the first opening is sealingly closed by the first door and an opening position into which the first door frees the first opening; and
an actuation system configured to be mechanically coupled to the first door to move the first door between the closing position and the opening position and/or between the opening position and the closing position,
wherein the actuation system is configured to disengage from the first door when the first door moves toward the closing position and a load applied to the first door in opposition to the motion toward the closing position is equal or greater than a threshold value.

2. The machine for treating a medical apparatus of claim 1, wherein the actuation system comprises:
a main actuator comprising a shaft adapted to move according to the first direction; and
at least one first electromagnetic lock operatively connected to the shaft of the main actuator, the first electromagnetic lock being configured to be selectively locked with a counter element of the first door,
wherein the first electromagnetic lock is configured to be unlocked from the counter element of the first door during a motion of the first door toward the closing position when a load applied to the first door in opposition to the motion toward the closing position is equal or greater than the threshold value.

3. The machine for treating a medical apparatus of claim 2, wherein the threshold value corresponds to a magnetic force locking the first electromagnetic lock to the counter element.

4. The machine for treating a medical apparatus of claim 2, wherein the actuation system is configured such that, when the first door moves toward the closing position and the load applied to the first door in opposition to the motion toward the closing position is equal or greater than the threshold value, the shaft is configured to pull the first electromagnetic lock away from the counter element thereby unlocking the first electromagnetic lock from the counter element.

5. The machine for treating a medical apparatus according of claim 2, wherein the first electromagnetic lock is configured to bear against the counter element of the first door according to the first direction such that the first door is pushed by the first electromagnetic lock for being moved toward the opening position and is pulled by the first electromagnetic lock for being moved toward the closing position.

6. The machine for treating a medical apparatus of claim 1, wherein the actuation system is automatic.

7. The machine for treating a medical apparatus of claim 1, wherein the first direction extends horizontally.

8. The machine for treating a medical apparatus of claim 1,
wherein the first treatment chamber comprises a second opening configured to allow a medical apparatus to be loaded into or unloaded from the enclosure, and
the machine for treating a medical apparatus further comprises a second door configured to be moved horizontally between a closing position into which the second opening is sealingly closed by the second door and an opening position into which the second door frees the second opening, the actuation system being configured be mechanically coupled to the second door to move the second door independently between the closing position and the opening position, wherein the actuation system is configured to disengage from the second door when the second door moves toward the closing position and a load applied to the second door in opposition to the motion toward the closing position is equal or greater than the threshold value.

9. The machine for treating a medical apparatus of claim 8, wherein the actuation system comprises a second electromagnetic lock operatively connected to the shaft of the main actuator, the second electromagnetic lock is configured to be selectively locked with a counter element of the second door, wherein the second electromagnetic lock is configured to be unlocked from the counter element of the second door during a motion of the second door toward the closing position when a load applied to the second door in opposition to the motion toward the closing position is equal or greater than the threshold value.

10. The machine for treating a medical apparatus of claim 1, further comprising at least one first treatment circuit adjacent to the first treatment chamber along the first direction which is adapted to treat a medical apparatus disposed in the first treatment chamber by immersion and/or by spraying a cleaning and/or disinfectant solution taken from a reservoir.

11. The machine for treating a medical apparatus of claim 1, further comprising a frame, the frame comprising at least one first rail extending according to the first direction into which the first door slides between the closing position and the opening position.

12. The machine for treating a medical apparatus of claim 11, wherein the frame is adapted to be embedded in a wall separating a controlled atmosphere enclosure from an enclosure with a non-controlled atmosphere.

13. The machine for treating a medical apparatus of claim 11, wherein the at least one first door is configured to be locked with the frame when in the closing position.

14. The machine for treating a medical apparatus of claim 1, further comprising a programmable controller for controlling the actuation system to open and/or close one or more of the doors of the machine.

15. The machine for treating a medical apparatus of claim 1, further comprising a user interface to allow an operator to actuate one or more of the doors of the machine.

16. The machine for treating a medical apparatus of claim 1, wherein the machine is an endoscope reprocessing machine and the medical apparatus is an endoscope.

17. A method comprising:
opening a door to provide access to a treatment chamber of an endoscope reprocessing machine;
inserting an endoscope into the treatment chamber through the door;
causing an actuation system mechanically coupled to the door to move the door between an opening position and a closing position; and
disengaging the actuation system from the door in response to a load applied to the door in opposition to motion toward the closing position being equal or greater than a threshold value.

18. The method of claim 17, wherein:
the actuation system is mechanically coupled to the door via an electromagnetic lock; and
disengaging the actuation system from the door comprises unlocking the electromagnetic lock during motion of the door toward the closing position when the load applied to the door in opposition to the motion toward the closing position is equal or greater than the threshold value.

19. The method of claim 17, wherein the actuation system is automatic and causing the actuation system to move the door comprises causing the actuation system to automatically move the door.

20. The method of claim 17, wherein causing the actuation system to move the door comprises receiving a command via a user interface.
